(19) United States
(12) Patent Application Publication
(10) Pub. No.: US 2008/0025523 A1
(43) Pub. Date: Jan. 31, 2008

(54) SYSTEM AND METHOD FOR NOISE CANCELING IN A MOBILE PHONE HEADSET ACCESSORY

(75) Inventor: Douglas Miller, Raleigh, NC (US)

Correspondence Address: MOORE AND VAN ALLEN PLLC FOR SEMC, P.O. BOX 13706, 430 DAVIS DRIVE, SUITE 500, RESEARCH TRIANGLE PARK, NC, 27709, US

(73) Assignee: SONY ERICSSON MOBILE COMMUNICATIONS AB, Lund (SE)

(21) Appl. No.: 11/460,655

(22) Filed: Jul. 28, 2006

(51) Int. Cl. A61F 11/06; H04R 1/10; G10K 11/16; H03B 29/00

(52) U.S. Cl. 381/71.6; 381/74

(57) ABSTRACT

Disclosed is a system, method, and computer program product for canceling ambient noise in a portable mobile communications device that is coupled with a standard non-noise canceling headset assembly. The portable mobile communications device receives detected ambient noise from the headset assembly microphone and creates a noise canceling ambient noise sound wave that is 180° out of phase with the ambient noise sound wave. The noise canceling ambient noise sound wave is then combined with an intended sound wave such as music or speech. The combined noise canceling ambient noise sound wave and intended sound wave are then forwarded to the headset assembly such that the noise canceling ambient noise sound wave negates the ambient noise present at the headset assembly during playback of the intended sound wave. Noise canceling can be achieved using hardware filters or via a noise canceling software application within the portable mobile communications device.

### BACKGROUND OF THE INVENTION

Portable mobile communications devices such as mobile phones are becoming more sophisticated and include many new features and capabilities. One of the most popular and safety oriented portable mobile communication device accessory is a headset. A headset allows the user to place and receive calls without having to hold the portable mobile communication device against the ear. This frees the user's hands for other tasks and is especially beneficial when the user is driving an automobile.

Standard headsets employ ear buds that are inserted into the ear canal or cover the opening to the ear canal to pass sound waves to the user along with a microphone to pick up the user's voice. Often the user may find himself in a noisy ambient environment that detracts from the effectiveness of the headset.

Noise canceling headsets are somewhat commonly known but can prove to be somewhat expensive or overly cumbersome. Noise canceling is achieved by using the headset microphone to pick up ambient noise around the headset area. The ambient noise, in the form of a sound wave, is then processed to create a duplicate sound wave that is 180° out of phase. This out of phase ambient noise sound wave is then combined with the intended sound wave (i.e., music or voice) to be played back by the headset with the expectation that the out of phase ambient noise sound wave will cancel out the actual ambient noise sound wave leaving only the intended souond wave for the user to hear via the headset speakers.

The processing and equipment needed to create the out of phase (noise canceling) sound wave takes up space in the headset assembly making the headset more expensive and cumbersome to the user.

What is needed is a software or hardware solution that can effectively apply noise canceling techniques to standard portable mobile communication device headsets without having to perform the noise canceling procedures in the actual headset.

### BRIEF SUMMARY OF THE INVENTION

Disclosed is a system, method, and computer program product for canceling ambient noise in a portable mobile communications device that is coupled with a standard non-noise canceling headset assembly. The portable mobile communications device receives detected ambient noise from the headset assembly microphone and creates a noise canceling ambient noise sound wave that is 180° out of phase with the ambient noise sound wave. The noise canceling ambient noise sound wave is then combined with an intended sound wave such as music or speech. The combined noise canceling ambient noise sound wave and intended sound wave are then forwarded to the headset assembly such that the noise canceling ambient noise sound wave negates the ambient noise present at the headset assembly during playback of the intended sound wave. Noise canceling can be achieved using hardware filters or via a noise canceling software application within the portable mobile communications device.

### BRIEF DESCRIPTION OF THE DRAWINGS

FIG. 1 is a block diagram of a portable mobile communications device capable of interacting with a wired and wireless headset according to the present invention.

FIG. 2 is a block diagram of portable mobile communications device components used to implement the present invention.

FIG. 3 is a flowchart describing a hardware implemented noise canceling process in a portable mobile communications device in accordance with an embodiment of the present invention.

FIG. 4 is a flowchart describing a software implemented noise canceling process in a portable mobile communications device in accordance with an embodiment of the present invention.

### DETAILED DESCRIPTION OF THE INVENTION

The present invention shifts noise canceling components and processing from the headset itself to the portable mobile communications device. The only component of the headset that is needed and used to perform noise canceling is the microphone. Thus, the present invention has the ability to provide any headset with noise canceling characteristics meaning the user is spared the expense and added bulk of noise canceling headsets. The techniques disclosed and claimed herein are equally applicable to hard-wired headset configurations that connect directly to the portable mobile communication device and wireless headset configurations such as the now ubiquitous Bluetooth™ headsets.

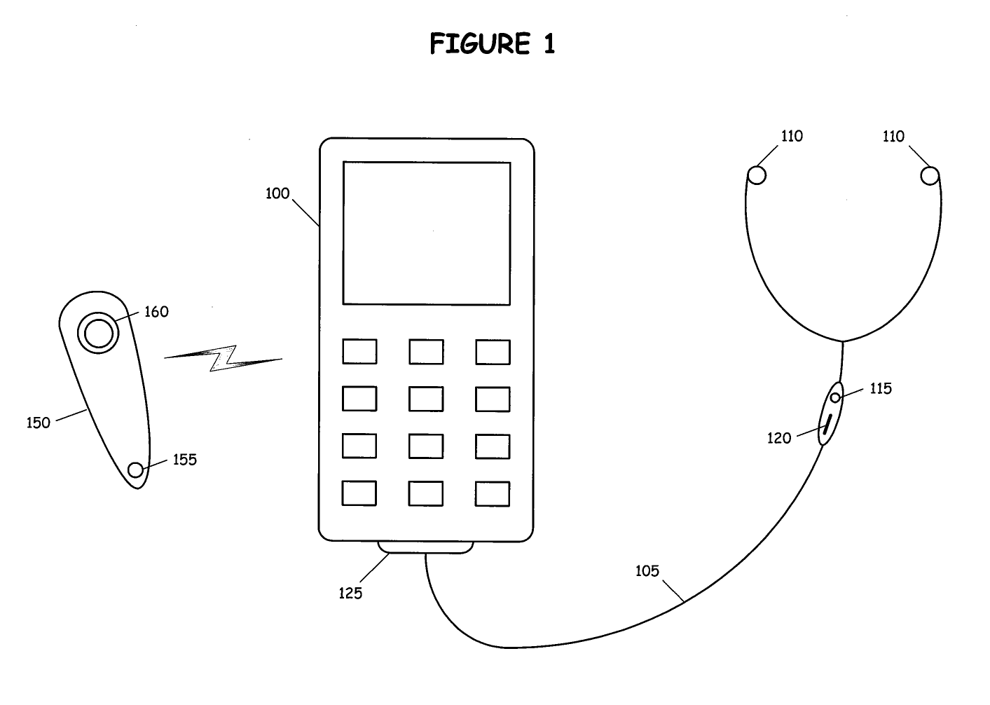

FIG. 1 is a block diagram of a portable mobile communications device 100 capable of interacting with a wired 105 and wireless 150 headset according to the present invention.

The wired headset assembly 105 includes a pair of ear buds 110 that contain speakers for producing sound waves and a microphone 115 for picking up sound waves to be reproduced elsewhere. A coupling 125 provides a physical interface between the headset assembly 105 and the portable mobile communications device 100 that allows electrical signals to be exchanged between the two devices. A control button 120 is also shown on the headset assembly 105 that acts to control the functions of the headset assembly such as connecting or terminating a phone call.

The wireless headset assembly 150 includes a microphone component 155 and a speaker component 160. Not illustrated, but contained within the wireless headset assembly 150, is the RF circuitry that interfaces with the portable mobile communications device 100. The wireless headset assembly 150 is typically of the Bluetooth™ type that are common to many portable mobile communications devices today. However, the present invention is not limited to Bluetooth™ type wireless headsets since the techniques described herein are independent of the RF carrier circuitry employed to communicate with the portable mobile communications device 100. Thus, any wireless protocol would likely suffice.

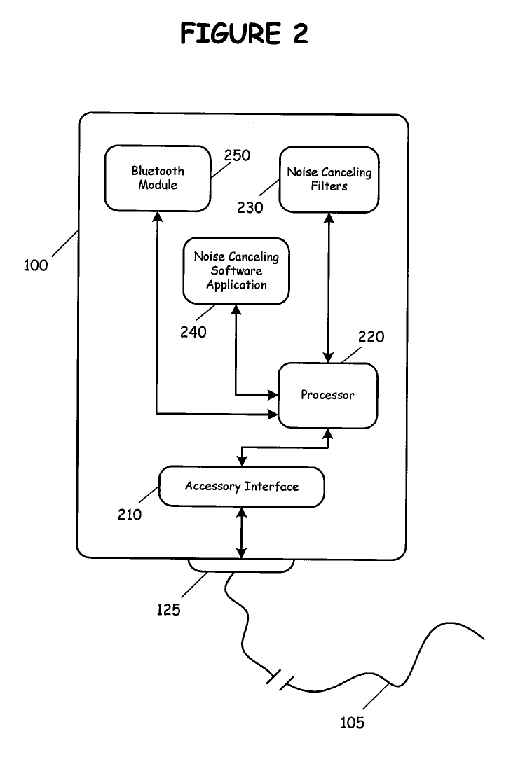

FIG. 2 is a block diagram of portable mobile communications device components used to implement the present invention. The portable mobile communications device 100 includes an accessory interface component 210 for exchanging signals with accessories that are ‘plugged’ into the portable mobile communications device 100. One such accessory can be a wired headset assembly 105. The accessory interface component 210 is coupled with a processor 220 that receives and processes signals to and from the accessory interface component 210. The processor 220, in turn, is coupled with, among other components, noise canceling filters 230 (for a hardware implementation), a noise canceling application 240 (for a software implementation), and a Bluetooth™ module 250 (for a wireless headset implementation).

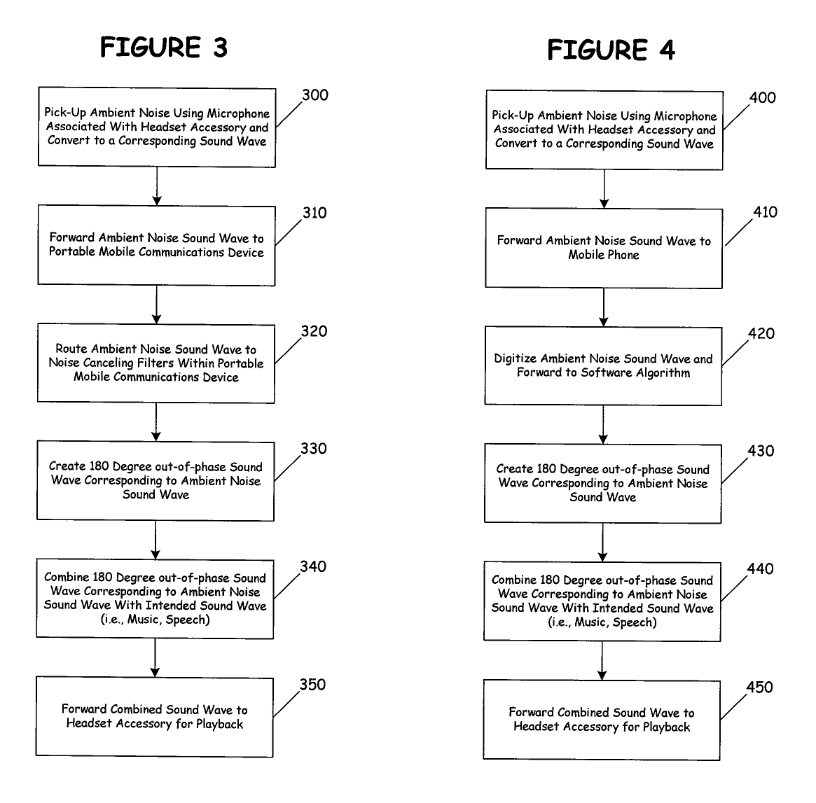

FIG. 3 is a flowchart describing a hardware implemented noise canceling process in a portable mobile communications device in accordance with an embodiment of the present invention. The processes described in the flowchart of FIG. 3 apply equally to wired and wireless headset implementations.

The first step is for the headset assembly microphone to pick up ambient noise and convert it to an ambient noise sound wave 300. The ambient noise sound wave is then forwarded to the portable mobile communications device 310 via the accessory interface component or Bluetooth™ module. The ambient noise sound wave is then routed to noise canceling filters within the portable mobile communications device 320 via the processor. The noise canceling filters create a duplicate 180° out of phase ambient noise sound wave 330. The out of phase ambient noise sound wave is then combined with whatever sound wave is intended (i.e., music, speech) for the headset assembly 340. The combine sound wave is then forwarded to the headset accessory for playback 350. The out of phase ambient noise sound wave should cancel with the actual ambient noise sound wave leaving the intended sound wave as the dominant audio perceived by the user.

FIG. 4 is a flowchart describing a software implemented noise canceling process in a portable mobile communications device in accordance with an embodiment of the present invention. Again, the processes described in the flowchart of FIG. 4 apply equally to wired and wireless headset implementations.

Again, the first step is for the headset assembly microphone to pick up ambient noise and convert it to an ambient noise sound wave 400. The ambient noise sound wave is then forwarded to the portable mobile communications device 410 via the accessory interface component or Bluetooth™ module. The ambient noise sound wave is then digitized routed to a noise canceling software application within the portable mobile communications device 420 via the processor. An algorithm in the noise canceling software application then creates a duplicate 180° out of phase ambient noise sound wave 430. The out of phase ambient noise sound wave is then combined with whatever sound wave is intended (i.e., music, speech) for the headset assembly 440. The combine sound wave is then forwarded to the headset accessory for playback 450. As previously described, the out of phase ambient noise sound wave should cancel with the actual ambient noise sound wave leaving the intended sound wave as the dominant audio perceived by the user.

A software implementation provides another advantage in that the user can exercise additional control over the noise canceling sound wave created. The user can digitally identify and clip the noise canceling signal to preserve certain background noise such as emergency vehicle sirens or the human voice. This would be part of the noise canceling software application and involve identifying specific frequency ranges to exclude from noise canceling.

The actual noise canceling technique of creating a 180° out of phase duplicate ambient noise sound wave is well known in the art for both hardware filters and a digitized software application. Moreover, combining the 180° out of phase duplicate ambient noise sound wave with an intended sound wave (speech or music) is also well known in the art. The present invention is not concerned with noise canceling techniques per se but rather in the ability to perform noise canceling procedures separate from a headset assembly. This allows any headset assembly having a microphone to achieve noise canceling not just specialized noise canceling headset assemblies that are more expensive and cumbersome.

As will be appreciated by one of skill in the art, the present invention may be embodied as a method, system, or computer program product. Accordingly, the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present invention may take the form of a computer program product on a computer-usable storage medium having computer-usable program code embodied in the medium.

Any suitable computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Computer program code for carrying out operations of the present invention may be written in an object oriented programming language such as Java, Smalltalk, C++ or the like. However, the computer program code for carrying out operations of the present invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

The present invention is described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems which perform the specified functions or acts, or combinations of special purpose hardware and computer instructions.

Although specific embodiments have been illustrated and described herein, those of ordinary skill in the art appreciate that any arrangement which is calculated to achieve the same purpose may be substituted for the specific embodiments shown and that the invention has other applications in other environments. This application is intended to cover any adaptations or variations of the present invention. The following claims are in no way intended to limit the scope of the invention to the specific embodiments described herein.

1. A method of canceling ambient noise in a portable mobile communications device that is coupled with a standard non-noise canceling headset assembly, the method comprising:
receiving detected ambient noise from the headset assembly microphone;
creating a noise canceling ambient noise sound wave that is 180° out of phase with the ambient noise sound wave;
combining the noise canceling ambient noise sound wave with an intended sound wave; and
forwarding the combined noise canceling ambient noise sound wave and intended sound wave to the headset assembly such that the noise canceling ambient noise sound wave negates the ambient noise present at the headset assembly during playback of the intended sound wave.

2. The method of claim 1 wherein the step of creating a noise canceling ambient noise sound wave that is 180° out of phase with the ambient noise sound wave is achieved using a hardware noise canceling filter.

3. The method of claim 1 wherein the step of creating a noise canceling ambient noise sound wave that is 180° out of phase with the ambient noise sound wave is achieved using a noise canceling software application that digitizes the ambient noise sound wave and constructs a corresponding noise canceling ambient noise sound wave that is 180° out of phase with the digitized ambient noise sound wave.

4. The method of claim 3 further comprising identifying a frequency band to be excluded from the corresponding noise canceling ambient noise sound wave that is 180° out of phase with the digitized ambient noise sound wave such that ambient noise within the identified frequency band is not subject to noise canceling.

5. A system for canceling ambient noise in a portable mobile communications device that is coupled with a standard non-noise canceling headset assembly, the method comprising:
means for receiving detected ambient noise from the headset assembly microphone;
means for creating a noise canceling ambient noise sound wave that is 180° out of phase with the ambient noise sound wave;
means for combining the noise canceling ambient noise sound wave with an intended sound wave; and
means for forwarding the combined noise canceling ambient noise sound wave and intended sound wave to the headset assembly such that the noise canceling ambient noise sound wave negates the ambient noise present at the headset assembly during playback of the intended sound wave.

6. The system of claim 5 wherein the means for creating a noise canceling ambient noise sound wave that is 180° out of phase with the ambient noise sound wave includes a hardware noise canceling filter.

7. The system of claim 5 wherein the means for creating a noise canceling ambient noise sound wave that is 180° out of phase with the ambient noise sound wave includes a noise canceling software application that digitizes the ambient noise sound wave and constructs a corresponding noise canceling ambient noise sound wave that is 180° out of phase with the digitized ambient noise sound wave.

8. The system of claim 7 further comprising means for identifying a frequency band to be excluded from the corresponding noise canceling ambient noise sound wave that is 180° out of phase with the digitized ambient noise sound wave such that ambient noise within the identified frequency band is not subject to noise canceling.

9. A computer program product embodied on a computer readable storage medium for canceling ambient noise in a portable mobile communications device that is coupled with a standard non-noise canceling headset assembly, the computer program product comprising:
computer program code for receiving detected ambient noise from the headset assembly microphone;
computer program code for creating a noise canceling ambient noise sound wave that is 180° out of phase with the ambient noise sound wave;
computer program code for combining the noise canceling ambient noise sound wave with an intended sound wave; and
computer program code for forwarding the combined noise canceling ambient noise sound wave and intended sound wave to the headset assembly such that the noise canceling ambient noise sound wave negates the ambient noise present at the headset assembly during playback of the intended sound wave.

10. The computer program product embodied on a computer readable storage medium of claim 9 wherein the computer program code for creating a noise canceling ambient noise sound wave that is 180° out of phase with the ambient noise sound wave is achieved using computer program code for directing the ambient noise sound wave to a hardware noise canceling filter.

11. The computer program product embodied on a computer readable storage medium of claim 9 wherein the computer program code for creating a noise canceling ambient noise sound wave that is 180° out of phase with the ambient noise sound wave is achieved using computer program code that digitizes the ambient noise sound wave and constructs a corresponding noise canceling ambient noise sound wave that is 180° out of phase with the digitized ambient noise sound wave.

12. The computer program product embodied on a computer readable storage medium of claim 11 further comprising computer program code for identifying a frequency band to be excluded from the corresponding noise canceling ambient noise sound wave that is 180° out of phase with the digitized ambient noise sound wave such that ambient noise within the identified frequency band is not subject to noise canceling.
